(19) United States
(12) Patent Application Publication
(10) Pub. No.: US 2013/0064870 A1
(43) Pub. Date: Mar. 14, 2013

(54) DRY POWDER INHALATION COMPOSITION

(71) Applicants: Subhas Bhowmick, Baroda (IN); Prashant Kane, Baroda (IN); Ganesh S, Baroda (IN); Tarun Patel, Baroda (IN); Swapnil Chudiwal, Baroda (IN)

(72) Inventors: Subhas Bhowmick, Baroda (IN); Prashant Kane, Baroda (IN); Ganesh S, Baroda (IN); Tarun Patel, Baroda (IN); Swapnil Chudiwal, Baroda (IN)

(73) Assignee: SUN PHARMA ADVANCED RESEARCH COMPANY LIMITED, Mumbai, Maharashtra (IN)

(21) Appl. No.: 13/699,130

(22) PCT Filed: May 13, 2011
(86) PCT No.: PCT/IN2011/000337

§ 371 Date: Nov. 20, 2012

(30) Foreign Application Priority Data
May 20, 2010 (IN) 1581/MUM/2010

(51) Int. Cl. A61K 9/14; A61K 31/56; A61P 29/00; A61P 11/00; A61K 31/205; A61P 11/06

(52) U.S. Cl. 424/400; 514/555; 514/171

(57) ABSTRACT

The dry powder inhalation composition comprising
(1) salmeterol xinafoate having mean particle size in range of 2.0μ-6μ microns and a tapped density in the range of 0.20 g·cm⁻³ to 0.45 g·cm⁻³ and
(2) optionally, one or more other active ingredients
   and pharmaceutically acceptable carrier.

The present invention relates to dry powder inhalation composition for the administration of medicament to patients.

### BACKGROUND OF THE INVENTION

Pulmonary drug delivery has been gaining considerable interest as an effective and convenient alternative route of dry administration. Dry powder inhalers (DPI's) are well known devices for administering pharmaceutically active agents to the respiratory tract. Dry powder inhalation compositions for use as inhalable medicaments in DPI's typically comprises a pharmaceutically active agent intimately admixed with an excess of pharmaceutically acceptable excipient or excipients (often referred to as carrier). Among the various active ingredients used to treat respiratory diseases such as asthma and COPD, salmeterol xinafoate in combination with steroids is well established. Changes in the particle size of medicament, is known to significantly affect its deposition to the lungs and therefore, affect the efficacy. Different factors play a role in functioning of the composition in terms of efficacy. Development of formulations in terms of particle size distribution, particle density, morphology, surface roughness, flowability and surface energy suitable for maximizing drug delivery to lung is of paramount importance for therapeutic DPIs. Also, for a drug like salmeterol, a localized action to lungs is desirable and it is important to note that no systemic action is desired. Further salmeterol xinafoate exhibits in two polymorphic forms and micronization leads to increased polymorph II which is also known to be unstable and more soluble as compared to polymorph I which is more stable and provides slower dissolution. The complexity further adds on when salmeterol is to be administered in combination with other drug, such as for eg. fluticasone propionate. This is because when these combinations are used in dry powder inhalers, the consistency of drug proportion in each dose cannot be easily controlled. The ratio of drugs in each dose significantly depends on the forces existing in each drug, between the drugs, between the drug and carrier material, and between the drug and the dry powder container of the inhaler device.

Salmeterol xinafoate in an easily fluidized crystalline form, with a dynamic bulk density of less than 0.1 g/cm³ which is either in the form of pure polymorph I or in the form of pure polymorph II is known in the prior art namely, U.S. Pat. No. 5,795,594 (referred to U.S. Pat. No. '594). The patent discloses the problem that the conventionally crystallized salmeterol xinafoate, even after micronization (fluid milling), exists in a form with poor flow characteristics. For example, it is cohesive and statically charged, which results in difficulties in handling the drug substance in pharmaceutical formulation processes. This U.S. Pat. No. '594 solves the above mentioned problem by describes subjecting salmeterol xinafoate to supercritical fluidization.

Henry et al in Pharmaceutical Research. Vol. 18, No. 6, 2001 discusses the problems associated with the process of micronization of salmeterol xinafoate such as micronized material resulting into highly charged, cohesive and difficult to process down-stream, and can even generate metastable solid phases and amorphous micro-domains leading to reduced stability and increased susceptibility to moisture.

Another patent literature, namely, PCT publication WO2009144193 describes a micronisable salmeterol xinafoate polymorph I characterized by a mean particle size between 5μ and 15μ and a bulk density between 0.1 and 0.2 g/ml. However, the publication discloses an unmicronized salmeterol xinafoate which is further required to be micronized to be suitable for incorporation into a dry powder inhalation composition.

Thus, there lies a need for a simple, economical yet simple process that is also feasible at large scale manufacturing of the micronized salmeterol xinafoate with specific polymorphic purity. We have found that when the salmeterol xinafoate with a controlled particle size, density and polymorphic form is formulated into a dry powder inhalation composition and is delivered to the lungs via a dry powder inhalation device, an unexpected improved efficacy is achieved. This is indeed surprising, because when certain DPI's were employed to deliver the salmeterol xinafoate of the present invention, equivalent efficacy of the inhaled active ingredient to the lungs was obtained only at its half of the total dose compared to the existing inhalation product. This means that there is a 50% dose reduction to arrive at the equivalent efficacy.

When the dry powder composition of the present invention comprising salmeterol xinafoate was tested in vitro by cascade impactor, using a dry powder inhalation device existing in the market, such as Rotahaler®, an improvement in the fine particle fraction was seen compared to the marketed product tested in the same device. This improvement, even though observed in vitro when tested in cascade impactor, can be attributed to the inventive dry powder inhalation composition comprising salmeterol xinafoate of specific tapped density, particle size and polymorphic purity.

### OBJECTS OF THE INVENTION

It is the object of the present invention to provide a dry powder inhalation composition which possesses predictable dissolution pattern and prolonged efficacy for once daily use.

It is also another object of the present invention to provide a dry powder inhalation composition comprising salmeterol xinafoate that provides a mass median aerodynamic diameter of particles (MMAD) in the range of 1 to 5.

Another object of the present invention is to provide a dry powder inhalation comprising salmeterol xinafoate which exerts equivalent efficacy at a reduced dose, compared to efficacy achieved by an existing product.

### SUMMARY OF THE INVENTION

The present invention provides a dry powder inhalation composition comprising
(1) salmeterol xinafoate having mean particle size in range of 2.0μ-6μ microns and a tapped density in the range of 0.20 g·cm⁻³ to 0.45 g·cm⁻³ and
(2) optionally, one or more other active ingredients
   and pharmaceutically acceptable carrier.

The present invention also provides a dry powder inhalation composition comprising salmeterol xinafoate obtained by a process comprising steps of
1. micronizing salmeterol xinafoate by milling
2. subjecting the micronized salmeterol xinafoate to a temperature of about 35° C. to 90° C. for a time period of about 1 hour to 120 hours, optionally, under pressure of about 1 to 100 bar.

The present invention also provides a method of treating asthma and other inflammatory respiratory disorders comprising administering a dry powder inhalation composition comprising salmeterol xinafoate and fluticasone propionate and wherein the method provides equivalent efficacy of the inhaled active ingredient to the lungs at half the total dose in comparison to the existing inhalation product.

### BRIEF DESCRIPTION OF THE DRAWINGS

FIG. 1 describes the scanning electron microscopic image of the salmeterol xinafoate particles indicating platelet structure.

FIG. 2—DSC data of the un-micronized salmeterol xinafoate indicating presence of a mixture of form I and form II in 88.8% and 11.20%, respectively

FIG. 3—DSC data of the micronized salmeterol xinafoate indicating mixture of polymorph I and polymorph II in 69.20% and 30.80%, respectively

FIG. 4—DSC data of the micronized salmeterol xinafoate subjected to conditioning micronized salmeterol xinafoate conditioned for 24 hours at 85° C., indicating mixture of polymorph I and polymorph II in 92.78% and 7.22%, respectively.

FIG. 5—DSC data of the micronized salmeterol xinafoate subjected to conditioning micronized salmeterol xinafoate conditioned for 40 hours at 85° C., indicating mixture of polymorph I and polymorph II in 96.40% and 3.66%, respectively.

FIG. 6—Changes in FEV1 in comparison with baseline value for test and reference in the function of time during the 24 hr observation period

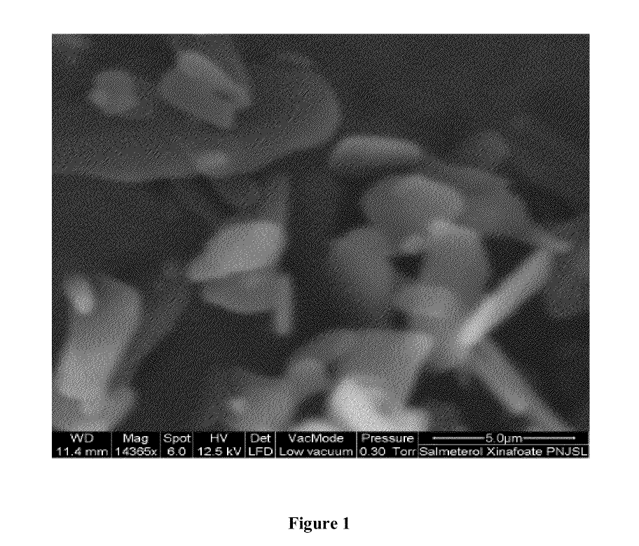

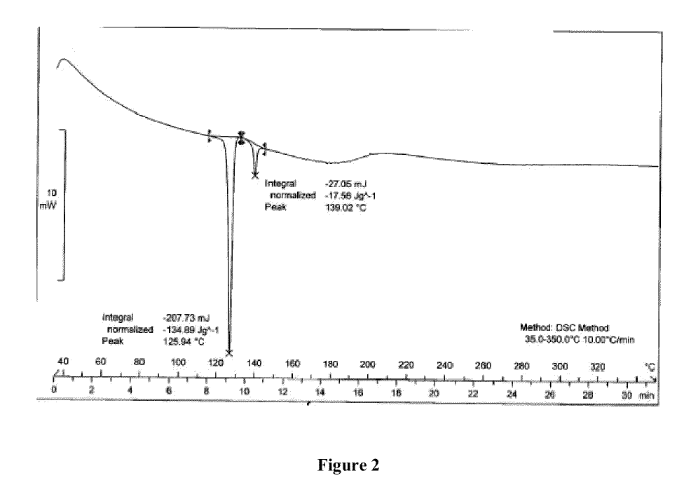

### DETAILED DESCRIPTION OF THE PRESENT INVENTION

The term ‘tapped density’ as used herein means that density attained after mechanically tapping a receptacle containing the powder sample.

The term ‘mean particle size’ as used herein means the particle size distribution of 50% of the population of the particles is less than the specified value. The particle size was measured using laser light diffraction method of particle size analysis.

The phrase ‘substantially free of polymorph II’ means the micronized salmeterol xinafoate having not more than 5%, preferably not more than 3%, most preferably not more than 1% of polymorph II. The polymorphic purity of the salmeterol xinafoate can be determined by any known analytical method. According to the present invention, the polymorphic purity is determined by differential scanning calorimeter (DSC). The melting points of polymorph I and polymorph II are distinct and this method can determine the presence of two.

A cascade impactor is a multi-stage sampling device for determining the size distribution of a particulate aerosol. The aerosol flows into the impactor whereupon it impinges upon a sequence of solids discs. Each disc (which represents a ‘stage’) is contained within a flow chamber and each chamber is connected in a vertical arrangement to the previous and next chamber in the sequence. Larger particles impact on the first disc and are captured. The sampling velocity increases for each successive chamber/disc so that successively smaller particles are collected. The final stage is typically a fine filter septum. Up to 10 stages are often used which divides the size distribution into an equivalent number of portions. The present invention may use eight Stage Sampler which meets the guidelines of the various world pharmacopoeias (e.g., United States Pharmacopoeia Chapter 601 “USP <601>”) to characterize metered-dose (MDI) and dry powder-dose inhalers (DPI), nebulizers, nasal sprays and other pulmonary drugs or a Next generation impactor (NGI) by Anderson. The testing of inhalation drugs goes hand and hand with cascade impactors, the size ranges collected are considered inhalable (generally <10 μm), just as the inhalation drugs should consistently arrive within the respiratory system into their target regions, the various stages represent the cut-off sizes when deposition may occur within the lungs.

The term ‘FEV1’ as used herein means a forced expiratory volume in 1 second (FEV1). This parameter represents the pharmacodynamic end point measure.

According to present invention, there is provided a dry powder inhalation composition comprising
(1) salmeterol xinafoate having mean particle size in range of 2.0μ-6μ and a tapped density in the range of 0.20 g·cm⁻³ to 0.45 g·cm⁻³ and
(2) optionally, one or more other active ingredients
   and pharmaceutically acceptable carrier.

In one embodiment of the present invention, the pharmaceutical composition salmeterol xinafoate ranging from about 5 μg to 50 μg, preferably 10 μg to 30 μg, most preferably about 25 micrograms per dose, equivalent to salmeterol base. In embodiments, when the dry powder inhalation composition comprises another active ingredient such as fluticasone propionate, it is present in amounts ranging from about 50 micrograms to 300 micrograms per dose.

In one embodiment, the present invention provides a dry powder inhalation composition comprising salmeterol xinafoate having mean particle size in range of 2.0μ-6μ and a tapped density in the range of 0.20 g·cm⁻³ to 0.45 g·cm⁻³. More particularly, the salmeterol xinafoate of the present invention is in the form of platelet shapes and a compressibility index in the range of about 30-60%, surface area ranging from about 5.0 to 8.0 (m2/g), a tapped density of 0.20 g·cm⁻³ to 0.45 g·cm⁻³ such that a mass mean aerodynamic diameter is achieved in the range of 2 to 4, preferably 3 to 4. In one preferred embodiment, the D₅₀ of the particles is about 3.0μ-4μ with a tapped density of 0.25 g·cm⁻³ to 0.35 g·cm⁻³. It may be noted that both these physical attributes of the salmeterol xinafoate of the present invention, are critical for providing an improved efficacy when the composition is delivered via an inhalation device to the lungs. This is postulated based on the in-vitro results when the composition was tested fine particle fraction and mass median aerodynamic diameter. The in-vitro results are provided in Example 2. The results indicate that salmeterol xinafoate having both the specified particle size and the specified tapped density provides a desirable mass median aerodynamic diameter and fine particle fraction.

According to present invention, there is provided a dry powder inhalation composition comprising
(1) salmeterol xinafoate having mean particle size in range of 2.0μ-6μ and a tapped density in the range of 0.20 g·cm⁻³ to 0.45 g·cm⁻³ and
(2) micronized fluticasone propionate
   and pharmaceutically acceptable carrier.

In one preferred aspect, the present invention provides a dry powder inhalation composition comprising salmeterol xinafoate having mean particle size in range of 2.0μ-6μ and micronized fluticasone propionate having a mean particle size in the range of 2.0μ-4μ and tapped density of 0.20 g·cm⁻³ to 0.45 g·cm⁻³. More particularly, the salmeterol xinafoate of the present invention is in the form of platelet shapes and a compressibility index in the range of about 30%-60%, surface area ranging from about 5.0 to 8.0 (m2/g), a tapped density of 0.20 g·cm⁻³ to 0.45 g·cm⁻³ such that a mass mean aerodynamic diameter is achieved in the range of 2 to 4, preferably 3 to 4.

It is another aspect of the present invention to provide a process for preparing the substantially pure salmeterol xinafoate polymorph I that is free of polymorph II. The process comprising the steps of
micronizing salmeterol xinafoate by milling
subjecting the micronized material to a temperature of about 35° C. to 90° C. and optionally, applying a pressure of about 1 to 100 bar for a time period of about 1 hour to 120 hours.

The micronization of salmeterol xinafoate is carried out with the exclusion of moisture, more preferably using an atmosphere such as atmospheric air, nitrogen or carbon dioxide. Preferable micronization is carried out by air jet mills in which the material is commuted by the impact of the particles on one another and on the walls of the grinding container. The material for grinding is conveyed by the grinding gas under specific pressures (grinding pressure). The grinding pressure is about 1 and 5 bars. The material for grinding is fed into the air jet mill by means of the feed gas at a feed pressure of 2-10 bars and fed rate of 0.1-10 g/min. The temperature of the inert gas is set at around 10° C.-30° C. The micronized salmeterol xinafoate is then subjected to a conditioning process which involves exposing the micronized salmeterol xinafoate particles to a combination of process variables such as temperature, pressure and optionally gas environment such as air, or inert gases such as nitrogen or carbon dioxide for pre-defined time interval so that a polymorphic form 1 substantially free of polymorphic form 2 is obtained. It was observed that the temperature range could be adjusted by increasing the pressure, to achieve the same effect. Thus, by varying these process parameters, it was found to provide a micronized salmeterol xinafoate, with desirable polymorphic purity.

In one embodiment of the present invention, an unmicronized salmeterol xinafoate was subjected to jet milling to get micronized salmeterol xinafoate of specific desirable particle size range, for example having mean particle size in range of 2.0μ-6μ. These micronized salmeterol xinafoate particles are subjected to a conditioning by exposing them at a specific temperature, pressure and time cycle. Preferably, the conditioning is carried out in a hot air oven. However, any other suitable means can be employed so that the temperature and pressure is maintained for long duration of hours like in few days. It may be possible to carry out this process in an autoclave in an inert atmosphere like carbon dioxide or nitrogen gas. It may be possible to carry out this in a pressurized vessel.

In one embodiment, the parameters for conditioning included a temperature range of about 35 to about 90° C. and pressure of about 100 bar under an inert gas atmosphere using inert gases such as N₂, CO₂ for a period of 1 to 120 hours to obtain salmeterol xinofoate with polymorph I and substantially free of polymorph II. In one preferred process, the conditioning of the micronized salmeterol xinafoate was done at 40° C. and 80 bar pressure in carbon dioxide environment for 80 hours. Following this process, it was surprisingly found that the micronized salmeterol xinafoate showing presence of a mixture of polymorph I and II, in 44.18% and 34.34% before conditioning, showed a drastic reduction in polymorph II percentage to 9.09%. In another set of process variables, such as conditioning for 24 hours at 85° C. or 40 hours at 85° C., it was found to provide a polymorph II to amounts as low as 7.22% and 3.60%, respectively.

In one preferred embodiment, the process involved steps of
1. milling salmeterol xinafoate using a jet mill at a grinding pressure of about 1-5 bars, feed pressure of about 2-10 bars, feed rate of 0.1-10 g/min, where in temperature of gas used for micronization is 10° C. to 30° C.
2. subjecting the micronized material to a temperature of about 35° C. to 90° C. and optionally, applying a pressure of about 1 to 100 bar for a time period of about 1 hour to about 50 hours.

The inventors have surprisingly found that when the salmeterol xinafoate was subjected to the process of micronization followed by conditioning at a temperature of about 50° C. to 90° C. for about 1-5 days without application of pressure, the salmeterol xinafoate obtained was substantially free of polymorph II. By the term ‘substantially free of polymorph II’ as used herein means that the salmeterol xinafoate in the form of polymorph II is not present in amount more than 5%.

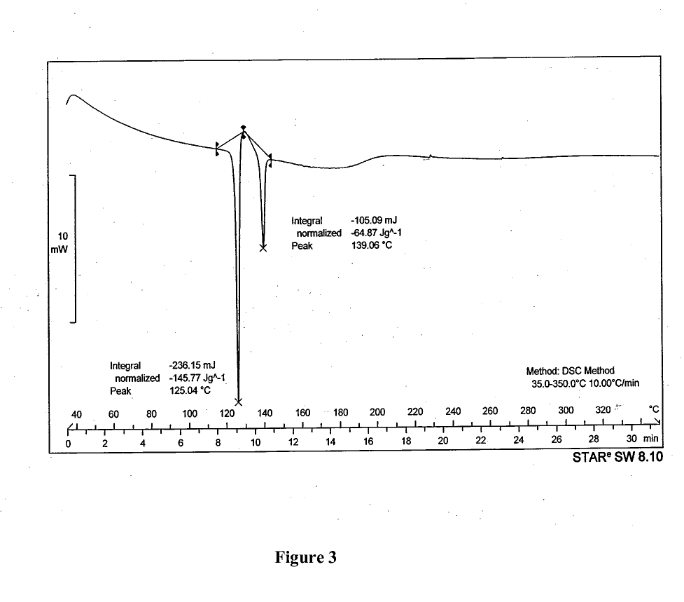

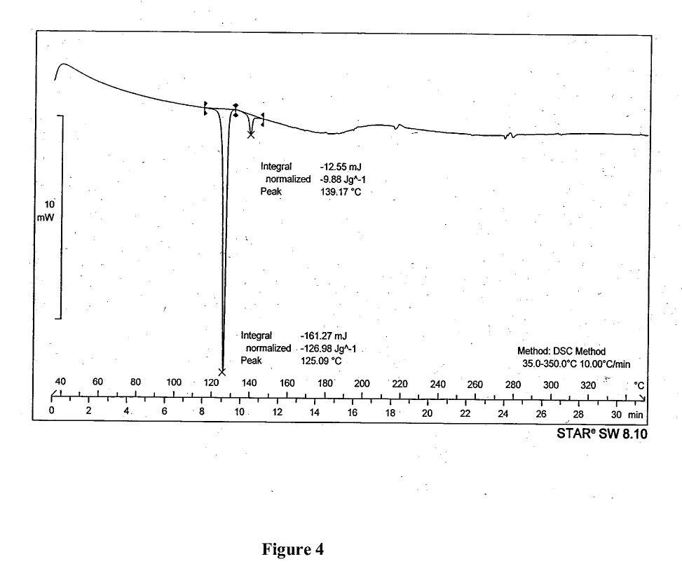

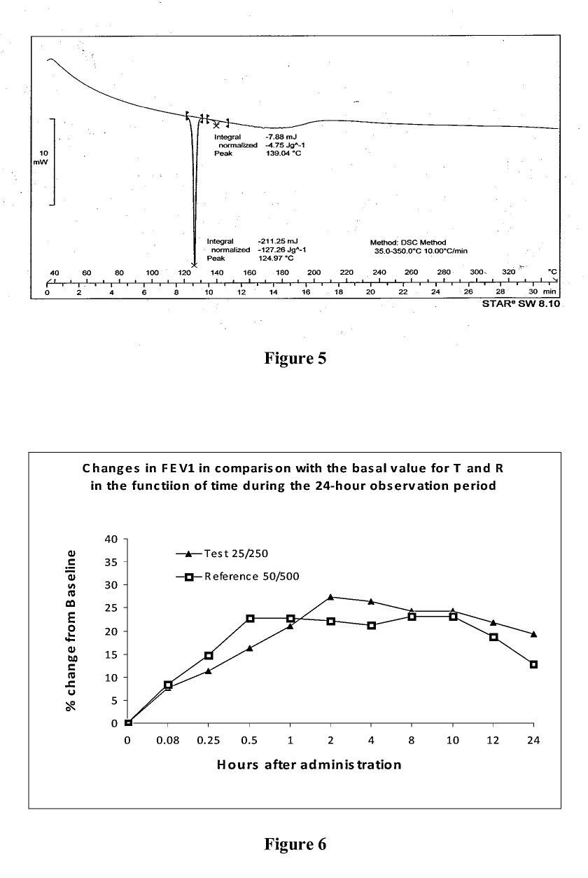

Particularly, it was observed that the un-micronized salmeterol xinafoate indicating presence of a mixture of form I and form II in 88.8% and 11.20%, respectively. It is known and observed that when such an unmicronized material is subjected to micronization, uncontrolled generation of the polymorph II occurred. This was observed and is presented in FIG. 3 where the DSC data of the micronized salmeterol xinafoate shows a mixture of polymorph I and polymorph II in 69.20% and 30.80%, respectively. When the micronized material is further subjected to conditioning as claimed in the present invention, the polymorph II contents were found to be present in limits. For example, FIG. 4 shows a DSC data of the micronized salmeterol xinafoate which was subjected to conditioning process of the present invention, conditioning being done for 24 hours at 85° C., the material showed low levels of polymorph II content of about 7.22%. It is therefore possible to control the polymorph II content by varying the conditioning parameters. For example, when the micronized salmeterol xinafoate was subjected to conditioning micronized salmeterol xinafoate for 40 hours at 85° C., a very low polymorph II content of only 3.66% was seen (FIG. 5).

Thus, the invention have provided a simple, feasible at any may be said to solve the problem of requirement to use very tedious, expensive methods of making salmeterol xinafoate substantially pure polymorph I which have been reported in the literature. The process of the present invention is also industrially feasible and cost effective. The presence of polymorph II can be monitored by various techniques such as X-ray diffraction and differential scanning calorimetry.

In one embodiment, the salmeterol xinafoate was obtained by micronizing salmeterol xinafoate by milling in a jet mill with grinding parameters are as follows: grinding pressure: 1-5 bars, feed pressure: 2-10 bars, feed rate of 0.1-10 g/min, followed by subjecting the micronized material to a temperature of about 50° C. to 90° C., wherein the conditioning cycle is carried out for a time period o about 1 hour to 120 hours, preferably, 1 to 50 hours, most preferably, 20 hrs to 40 hrs. The polymorphic purity of the resultant salmeterol xinafoate so obtained was determined by recording the differential scanning calorimetry (See FIGS. 3-5).

Generally, the dry powder inhalation composition of the present invention comprises and pharmaceutically acceptable carriers that are known to be commonly used in the dry powder inhalation compositions. Particularly, the pharmaceutically acceptable carrier comprises a carbohydrate selected from the group consisting of fructose, glucose, mannitol, maltose, trehalose, cellobiose, lactose and sucrose wherein the carbohydrate is present in the form of a combination of fine and coarse particles. In one embodiment, the dry powder inhalation composition comprises fine particles of the carbohydrate are having a D₅₀ in the range of 3.0 μs to 7.0 μs and coarse particles of carbohydrate having D₅₀ in the range of 200.0 μs to 250.0 μs. The pharmaceutically acceptable vehicle of the dry powder inhalation composition of the present invention comprises one or more carbohydrates selected from the group consisting of fructose, glucose, mannitol, maltose, trehalose, cellobiose, lactose and sucrose wherein the carbohydrate is present in the form of a combination of fine and coarse particles. In one particular embodiment, the dry powder inhalation composition of the present invention comprises carbohydrates in the form of a combination of fine particles and coarse particles. The D₅₀ of the fine particles of the carbohydrate is in the range of 3.0 μs to 7.0 μs, whereas the D₅₀ of coarse particles of the carbohydrate is in the range of 200 μs to 250 μs. In one embodiment, the ratio between the fine particle and the coarse particles of a carbohydrate ranges from about 1 to 20% preferably 5-15%.

Yet another aspect of the present invention relates to providing a method of treatment of asthma and other inflammatory respiratory disorders comprising steps of administering by inhalation to humans in need of such treatment effective amounts of salmeterol or a physiologically salt of salmeterol or a solvate thereof, wherein said effective amounts are administered to the human in need together with a pharmaceutically acceptable carrier, wherein the medicament is administered by an inhalation device that enables higher amount of the inhaled drug to be delivered to the lungs.

Particularly, The present invention provides a method for the treatment of asthma and other inflammatory respiratory disorders which comprises administering by inhalation to humans in need of such treatment effective amounts of salmeterol or a physiologically salt of salmeterol or a solvate thereof, and fluticasone or a therapeutically salt of fluticasone or a solvate thereof, wherein said effective amounts are administered substantially simultaneously to the human in need, together with a pharmaceutically acceptable carrier, wherein the medicament is administered by an inhalation device that enables higher amount of the inhaled drug to be delivered to the lungs. Particularly, salmeterol xinafoate used in the method according to this embodiment is having a D₅₀ of about 3.0μ-4μ with a tapped density of 0.25 to 0.35 g·cm⁻³. Preferably, the salmeterol xinafoate is in the form of polymorph I, substantially free of polymorph II. In another embodiment, the tapped density of the salmeterol xinafoate ranges from 0.20 g·cm⁻³ to 0.40 g·cm⁻³ having mean particle size of about 5μ. The salmeterol xinafoate used in the dry powder inhalation composition of the present invention was found to provide mass mean aerodynamic diameter in the range of 2 to 4, preferably 3 to 4.

In one particular embodiment, it was found that when the average particle size of salmeterol xinafoate in the range of 1μ to 15μ, preferably 50% of the particles are less than 5 microns. Preferably, the salmeterol xinafoate is present as polymorph I that is substantially free of polymorph II, such a medicament having salmeterol xinafoate in the form of platelets, having mean particle size in range of 2.5-4.5, having a compressibility index in the range of about 30-60%, surface area ranging from about 5.0 to 8.0 (m2/g), a tapped density of 0.20 g·cm⁻³ to 0.40 g·cm⁻³ such that a mass mean aerodynamic diameter is achieved in the range of 2 to 4, preferably 3 to 4, was found to provide superior efficacy when administered to the lungs with the help of any inhalation device, particularly was found to provide higher amount of the drug to the lungs when administered using the device as disclosed in WO2009008001, which is incorporated by reference.

Particularly, when the dry powder inhalation device as disclosed in applicant's own patent publication, namely, WO2009008001 was used for the delivery of the dry powder inhalation composition of the present invention, the inhalation therapy was found to be superior in that it enables higher amount of the inhaled drug to be delivered to the lungs, making possible a considerable administered dose reduction. This was concluded from the comparative data, wherein a commercially available marketed composition of salmeterol when administered via other known dry powder inhalers available in the market. This conclusion is substantiated by the in vitro as given in Table 4 where an improvement in fine particle fraction as well as mass median aerodynamic diameter was seen when the composition was delivered through the same inhalation device that is the one described in WO2009008001 and tested in cascade impactor is well as in vivo data. The results of the in-vitro test reveal that the central deposition achieved from dry powder inhalation composition of the present invention was superior compared to the central deposition achieved by a commercially available dry powder inhalation composition comprising same active ingredient, but having double the dose compared to the pharmaceutical composition of the present invention. The evidence is provided in the Table 4. Particularly, not only the central deposition was improved, but the oropharyngeal deposition was reduced considerably by the administration of the composition of the present invention when administered by the device as disclosed in WO2009008001. This difference in the deposition achieved by the composition of the present invention was indeed surprising and unexpected. The data was promising because it showed possibility of a dose reduction. This was further confirmed by clinical trials in large number of human patients.

In order to evaluate the effect of the dry powder inhalation composition itself, the composition was delivered via two different inhalation devices namely, a marketed inhalation device, namely, such as for example Rotahaler® and the applicant's own patented device. The % fine particle fraction and mass mean aerodynamic diameter was determined by testing in vitro in cascade impactor. The therapeutic efficacy of the composition of the present invention in terms of fine particle fraction was determined by a cascade impaction method official in the United States Pharmacopoeia, chapter <601>. The results of the fine particle fraction indicated that the composition of the present invention achieved improved central deposition at reduced dose compared to the commercially available product which is described in U.S. Pat. No. RE 40,045. Surprisingly, not only the central deposition was enhanced but the peripheral deposition which is undesirable was significantly reduced (please refer to table 2 and table 3.

The results of this study are presented in the Table 6(a) and Table 6 (b). It was concluded that although patented device contributes to the superior efficacy, the dry powder inhalation composition of the present invention is also responsible for providing better fine particle fraction. This is concluded from the data given in table 6 (a) which shows an improved fine particle fraction achieved by dry powder pharmaceutical composition of the present invention when delivered by a Rotahaler® device. Rotahaler® product with its own inhalation composition provided a fine particle fraction of 12.3% compared to fine particle fraction of 24.8% obtained by the dry powder composition of the present invention, when delivered through the same Rotahaler® device. This was indeed a confirmatory test to see the effect of the composition of the present invention itself. Further, when the composition of the present invention was delivered via the applicant's patented inhalation device, a further improvement in fine particle fraction of 47.7% was seen. Thus, both the dry powder inhalation composition as well the applicant's own patented device, play a major role in providing improved efficacy, determined by in vitro testing, using cascade impactors. Without wishing to be bound by any theory, thus, applicants believe that the dry powder inhalation composition of the present invention will be contributing in providing improved efficacy, when administered in vivo.

According to the present invention, in one preferred embodiment, there is provided a method of treating patients having respiratory disorders by administration of the dry powder inhalation composition comprising salmeterol xinafoate with a controlled particle size, polymorphic form, shape and morphology and which has a tapped density in the range of 0.20 g·cm⁻³ to 0.30 g·cm⁻³ and in combination with fluticasone propionate and pharmaceutically acceptable carrier, wherein the medicament is administered by an inhalation device that enables higher amount of the inhaled drug to be delivered to the lungs.

In certain preferred embodiments, applicant's patented device described in PCT publication, WO2009008001 which is incorporated herein by reference is used for administering the novel dry powder inhalation composition of the present invention. The inhalation-activatable device as described in WO2009008001 for administration of medicament in powder form to the respiratory system of a user comprising a housing defining air inlet(s) and a mouthpiece, and a cap for covering the mouthpiece, wherein the housing contains a dose carrier with medicament in powder form arranged in plurality of dose units and a breath activated mechanism comprising an energy storing means, a triggering means, a piercing means and a reset means, in which the triggering means comprises a Breath Actuated Mechanism (BAM) flap mounted for movement between neutral and inward positions, the BAM flap being positioned away from the mouthpiece and substantially away from the air inlet(s) so as not to close the air inlet(s) in its neutral or inward position, such that inhalation through the mouthpiece causes movement of the BAM flap to its inward position, and other components movable relative to the position of the BAM flap such that when the BAM flap is in the inward position, a dose unit of the plurality of dose units on the dose carrier is punctured by a piercing means to deliver the medicament in powder form through the mouthpiece to the respiratory system of the user. Preferably, the inhalation-activatable device has the air passageway which comprises of a cyclone head, a conical region, a throat region and vanes at the end of the air passageway. Further, the inhalation-activatable device is designed such that the cyclone head, the conical region and the throat region are elliptical in cross section. In this embodiment, salmeterol xinafoate of the present invention is used in combination with fluticasone propionate.

Dry powder inhaler composition of the present invention packed in the applicant's own invented DPI device as disclosed in WO2009008001 gives equivalent efficacy as compare to commercially available products at 60% reduced dose levels. The composition according to the present invention was undertaken to compare pharmacodynamics of inhalation device containing Salmeterol 25 mcg/Fluticasone Propionate 250 mcg with commercially available product sold under the tradename of Seretide Accuhaler® (GSK) containing Salmeterol 50 mcg/Fluticasone Propionate 500 mcg. The results of this study show that composition comprising of salmeterol xinafoate results in prolonged efficacy. Percent change from baseline FEV1 is more than 15% till 24 hr for test product.

In one preferred embodiment, the present invention provides a method of treating patients having respiratory disorders wherein the dry powder inhalation composition is administered by use of the inhalation device as described in applicant's own PCT publication, WO2009008001 which is incorporated herein by reference. The inhalation-activatable device as described in WO2009008001 for administration of medicament in powder form to the respiratory system of a user comprising a housing defining air inlet(s) and a mouthpiece, and a cap for covering the mouthpiece, wherein the housing contains a dose carrier with medicament in powder form arranged in plurality of dose units and a breath activated mechanism comprising an energy storing means, a triggering means, a piercing means and a reset means, in which the triggering means comprises a Breath Actuated Mechanism (BAM) flap mounted for movement between neutral and inward positions, the BAM flap being positioned away from the mouthpiece and substantially away from the air inlet(s) so as not to close the air inlet(s) in its neutral or inward position, such that inhalation through the mouthpiece causes movement of the BAM flap to its inward position, and other components movable relative to the position of the BAM flap such that when the BAM flap is in the inward position, a dose unit of the plurality of dose units on the dose carrier is punctured by a piercing means to deliver the medicament in powder form through the mouthpiece to the respiratory system of the user. Preferably, the inhalation-activatable device has the air passageway which comprises of a cyclone head, a conical region, a throat region and vanes at the end of the air passageway. Further, the inhalation-activatable device is designed such that the cyclone head, the conical region and the throat region are elliptical in cross section.

The composition according to the present invention was undertaken to compare efficacy and safety of inhalation device containing Salmeterol 25 mcg/Fluticasone Propionate 250 mcg with commercially available product sold under the tradename of Seretide Accuhaler® (GSK) containing Salmeterol 50 mcg/Fluticasone Propionate 500 mcg. Results indicate an improvement in group treating by the method according to the present invention (S/FP 25/250 mcg) were similar to Seretide Accuhaler® group (S/FP 50/500 mcg). The FEV1 increased by 9.73% of predicted normal value in test group and 7.82% in reference group after 4-weeks of treatment. Although the S/FP 25/250 mcg group showed a trend towards more improvement than S/FP 50/500 mcg group in evening peak expiratory flow rate, all differences in subjective and objective outcome measures were not statistically significant. It may be concluded that the administered at half the dose of Seretide Accuhaler®, the efficacy achieved by the method of the present invention is not statistically significantly different on efficacy parameters evaluated.

Thus, the present invention further provides a method of treating asthma and other inflammatory respiratory disorders comprising administering a dry powder inhalation composition comprising salmeterol xinafoate and fluticasone propionate and wherein the method provides equivalent efficacy of the inhaled active ingredient to the lungs at half the total dose in comparison to the existing inhalation product.

While the invention has been described by reference to specific embodiments, this was done for purposes of illustration only and should not be construed to limit the spirit or the scope of the invention.

### Example 1

The micronized salmeterol xinofoate is then subjected to a conditioning process which involves exposing the micronized salmeterol xinafoate particles to a combination of temperature and optionally, pressure and/or inert gas environment for pre-defined time interval so that a polymorphic form 1 substantially free of polymorphic form 2 is obtained. According to this example, the micronized salmeterol xinafoate was subjected to higher temperature of about 85° C. for a period of 24 hour or 48 hours without application of pressure. In another process, a lower temperature of about 40° C. was set with a pressure of 80 bars under in CO₂ for 80 hours.

The results of the polymorphic purity were determined by differential scanning calorimeter and are tabulated below

TABLE 1
Process variation and polymorphic purity
of micronized salmeterol xinafoate

| | | | | % Salmeterol xinafoate | |
|---|---|---|---|---|---|
| Time | Pressure | Gas | Temperature | Polymorph | Polymorph |
| (hr) | (bar) | environment | (° C.) | form I | form II |
| 80 | 80 | CO₂ | 40 | 85.95 | 9.09 |
| 24 | — | Air | 85° C. | 92.78 | 7.22 |
| 40 | — | Air | 85° C. | 96.40 | 3.60 |

The conditioned micronized salmeterol xinafoate having mean particle size of 2.0-μ6μ microns and a tapped density in the range of 0.20 g·cm⁻³ to 0.45 g·cm⁻³ was tested for fine particle fraction, mass mean aerodynamic diameter in a cascade impactor. The composition was delivered using the applicant's patented device as described in the description. The results of the in-vitro testing are provided below in Table 2:

TABLE 2
Relation between tapped density and % FPF salmeterol
xinofoate obtained by process given in example 1.

| Mean | | | | | | | |
|---|---|---|---|---|---|---|---|
| particle | Tapped | | | | | | |
| size D₅₀ in | density | % FPF | | MMAD in μs | | GSD | |
| μms | (g/cm³ ) | Mean | SD | Mean | SD | Mean | SD |
| 5.99 | 0.43 | 20.91 | 0.872 | 4.199 | 0.083 | 1.734 | 0.010 |
| 4.02 | 0.25 | 41.115 | 0.876 | 3.165 | 0.034 | 1.747 | 0.000 |

From table 2 it may be observed that there is a significant effect of particle size and tapped density on the fine particle fraction as well as mass mean aerodynamic diameter. For example, the salmeterol xinafoate having average particle size of about 6 μms and having a tapped density of 0.43 g/cm³ although provides satisfactory mass mean aerodynamic diameter, the fine particle fraction is higher for salmeterol xinafoate having average particle size of about 4.02 μms and having a tapped density of 0.25.

This data establishes the fact that physical properties like the particle size and tapped density, play a critical role in providing therapeutic effect for medicament which is meant for inhalation.

### Example 2

The dry powder inhalation formulation contains the following ingredients

TABLE 2
composition of dry powder inhalation

| Ingredient | Quantity per blister (mcg) |
|---|---|
| Salmeterol xinafoate equivalent | 36.32 |
| to salmeterol 25 micrograms | |
| Fluticasone propionate | 250 |
| Lactose | q.s 10000 |

The salmeterol xinafoate used in the example 2 is prepared by the process of micronization and conditioning as described in the detailed description. Salmeterol xinafoate on mean particle size in range of 3μ to 4μ and tapped density in range of 0.25 g·cm⁻³ to 0.35 g·cm⁻³ was used. The fluticasone propionate was also micronized. The salmeterol xinafoate and fluticasone propionate and fine lactose particles were sieved through #200 in controlled temperature and humidity conditions. The coarse lactose particles were sieved through 40# in controlled temperature and humidity conditions. The sieved salmeterol xinafoate was mixed with the fine particle of lactose, whereas the sieved fluticasone propionate was mixed with the fine particles of lactose. A placebo blend of fine and coarse particles of lactose was done separately. The three pre-mixes were blended to get a final dry powder inhalation. The fine particle dose cascade impaction was determined for the dry powder inhalation composition packed in the applicant's own invented DPI device as disclosed in WO2009008001 in comparison to a commercially available dry powder inhalation composition of salmeterol xinafoate and fluticasone propionate.

The composition of dry powder inhalation was subjected to fine particle dose determination. This was determined according to the procedure given in USP chapter <601>. The fine particle fraction was measured in terms of deposition at various stages as per the pharmacopoeial method. The results of the amount of the salmeterol and fluticasone propionate dose from the commercially available product and that of the example 1 is tabulated in table 2. The results of the table 2 have been concluded in Table 3.

TABLE 3
In vitro cascade impactor data for deposition of salmeterol base
and fluticasone propionate from DPI of example 1 (25/250 mcg per
dose) and commercially available product (50/500 mcg per dose).

| | Commercially | | | |
|---|---|---|---|---|
| | available product | | Example 1 | |
| Fine particle | (50/500 mcg per dose) | | (25/250 mcg per dose) | |
| dose cascade | Salmeterol | Fluticasone | Salmeterol | Fluticasone |
| impaction (μgs) | base | propionate | base | propionate |
| Device | 0.00 | 0.00 | 0.00 | 0.00 |
| Throat | 6.27 | 62.99 | 6.18 | 44.37 |
| Preseperator | 27.91 | 253.99 | 2.49 | 32.72 |
| Stage 1 | 1.65 | 15.72 | 0.51 | 3.74 |
| Stage 2 | 2.24 | 25.04 | 2.96 | 19.24 |
| Stage 3 | 3.25 | 38.26 | 5.75 | 42.51 |
| Stage 4 | 2.71 | 32.45 | 4.25 | 43.69 |
| Stage 5 | 1.23 | 14.28 | 1.08 | 17.88 |
| Stage 6 | 0.37 | 4.28 | 0.19 | 4.64 |
| Stage 7 | 0.27 | 2.78 | 0.05 | 0.97 |
| filter | 0.16 | 1.70 | 0.04 | 0.89 |
| delivered dose | 46.06 | 451.11 | 23.50 | 210.65 |
| from the device | | | | |
| MMAD | 3.00 | 2.90 | 3.30 | 2.80 |
| Geometric SD | 2.20 | 2.10 | 1.60 | 1.70 |

### Example 3

The composition of Example 2 was compared with the commercially available formulation, in terms of central deposition (5-2 micron), peripheral deposition (<2 micron) and oropharyngeal deposition (>5 micron). The results are tabulated in Table 4 as follows:

TABLE 4
Comparison of in vitro deposition of the composition of the Example
1 with commercially available product using Cascade impactor

| | Commercially | | | |
|---|---|---|---|---|
| | available product | | Example 1 | |
| Fine particle | (50/500 mcg per dose) | | (25/250 mcg per dose) | |
| dose (μgs) | Salmeterol | Fluticasone | Salmeterol | Fluticasone |
| cascade impaction | base | propionate | base | propionate |
| Central | 5.61 | 66.08 | 9.80 | 78.23 |
| deposition | | | | |
| (5-2 micron) | | | | |
| Peripheral | 3.34 | 38.60 | 2.22 | 36.38 |
| deposition | | | | |
| (<2 micron) | | | | |
| Oropharyngeal | 34.18 | 316.68 | 8.67 | 77.09 |
| deposition | | | | |
| (>5 micron) | | | | |

It is known that the central deposition corresponds to the product efficacy. From the data presented in table 4, it is evident that dry powder inhalation composition of the present invention shows better central deposition of both, salmeterol xinafoate as well as fluticasone propionate compared to the commercially available product available under the brand name of Seretide Accuhaler 50/500. The peripheral and oropharyngeal deposition seems to be the minimum. The oropharyngeal deposition is known to be responsible for side effects caused by drug absorption via gastrointestinal tract, whereas the peripheral deposition is known to be responsible for side effects caused by drug absorption in systemic circulation.

### Example 4

The dry powder inhalation composition of the present invention was tested for fine particle fraction, mass median aerodynamic diameter using the cascade impactor described in the description. The data is presented in Table 5 below:

TABLE 5
Comparative MMDA, GSD and file particle dose (FPD)
(micrograms) across the three strengths
i.e. 25/50; 25/125 and 25/250 microgram/blister

| Drug | In vitro testing | | Strength | | | |
|---|---|---|---|---|---|---|
| substance | (n = 3) | | 25/50 | 25/125 | 25/250 | p value |
| Salmeterol | FPD (mcg) | Mean | 11.44 | 12.16 | 11.3 | 0.676 |
| | | SD | 0.594 | 1.710 | 1.146 | |
| | MMAD (μs) | Mean | 2.761 | 2.84 | 2.9 | 0.314 |
| | | SD | 0.05 | 0.183 | 0.079 | |
| | GSD | Mean | 1.707 | 1.713 | 1.677 | 0.780 |
| | | SD | 0.035 | 0.073 | 0.08 | |
| Fluticasone | FPD (mcg) | Mean | 19.95 | 49.9 | 90.250 | NA |
| propionate | | SD | 0.151 | 4.9 | 8.346 | |
| | MMAD (μs) | Mean | 2.493 | 2.515 | 2.563 | 0.606 |
| | | SD | 0.113 | 0.04 | 0.08 | |
| | GSD | Mean | 1.85 | 1.851 | 1.8 | 0.870 |
| | | SD | 0.074 | 0.074 | 0.095 | |

The values of FDP, MMAD and GSD for the different strengths of the salmeterol xinafoate and fluticasone show p value more than 0.05. This indicates no significant difference between the FDP, MMAD and GSD for salmeterol xinafoate and fluticasone propionate for the three dosage strengths. Thus, the dry powder inhalation composition of the present invention when administered via applicant's patented inhalation device, the salmeterol & fluticasone propionate are delivered with consistent MMAD, GSD & FPD across the three dosage levels indicating absence of any pharmaceutical interactions in combination product. To allow a controlled change between different doses consistency in MMAD, GSD & FPD across the three dosage levels is important. This was surprisingly and unexpected especially because the marketed products do not show such consistent performance across the strengths.

### Example 5

The dry powder inhalation composition of the present invention prepared as per the Example 2 delivered via applicant's own patented inhalation device was subjected to clinical trials to determine the efficacy and safety. The composition tested contained Salmeterol 25 mcg/Fluticasone Propionate 250 mcg delivered via applicant's patented device. The efficacy was compared with a commercially available product sold under the tradename of Seretide Accuhaler® (GSK) containing Salmeterol 50 mcg/Fluticasone Propionate 500 mcg.

The study was a 4-week, randomized, open-label, active-controlled, multicenter study (N=113) of patients aged 18-60 years with asthma, reversible bronchial obstruction, and FEV1 40-80% of the predicted normal. After washout from ongoing asthma treatments, there was a 2-week run-in period when patients were permitted use of rescue medication (Salbutamol metered dose inhaler). Patients who successfully competed the run-in period with FEV1 40-80% of the predicted normal were randomized 1:1 to device used in the method according to the present invention or Seretide Accuhaler® for a 4-week treatment period. Patients received twice daily inhalation between 8.00 a.m. to 10.00 a.m. and between 8.00 p.m. to 10.00 p.m. with approximately 12 hour interval between the two doses.

Results indicate an improvement in FEV in group treated by the method according to the present invention (S/FP 25/250 mcg) was similar to Seretide Accuhaler® group (S/FP 50/500 mcg). The FEV1 increased by 9.73% of predicted normal value in test group and 7.82% in reference group after 4-weeks of treatment. Although the S/FP 25/250 mcg group showed a trend towards more improvement than S/FP 50/500 mcg group in evening peak expiratory flow rate, all differences in subjective and objective outcome measures were not statistically significant.

It may be concluded that the administered at half the dose of Seretide Accuhaler®, the efficacy achieved by the method of the present invention is not statistically significantly different on efficacy parameters evaluated. It may be concluded that the dry powder inhalation composition when delivered to the lungs by applicant's patented inhalation device, equivalent efficacy at half amount of dose that of the commercially available dry powder inhalation product was achieved. Thus, the dry powder inhalation composition of the present invention can be said to achieve similar or prolonged efficacy at a lower dose level compared to the commercially available product.

### Example 6

The dry powder inhalation composition of the present invention (prepared as per example 1) when tested for fine particle fraction (% FPF) and GSD and MMAD in different inhalation devices, surprisingly it was found that composition provided improved drug delivery compared to the same composition delivered via another inhalation device. Thus, the composition can be said to contribute to the improved efficacy in terms of (% FPF) and GSD and MMAD. This is evident from the data given in Table 6 below:

TABLE 6a
Dry powder inhalation composition of the present invention delivered
via different inhalation devices

| Strength | | | | | | | | | | | | |
|---|---|---|---|---|---|---|---|---|---|---|---|---|
| Of | | | | | | | | | | | | |
| salmeterol | Composition delivered via | | | | | | Composition delivered via | | | | | |
| (SX) and | inhalation device available under | | | | | | applicant's patented | | | | | |
| fluticasone | the brand name of Rotahaler ® | | | | | | inhalation device by inventors | | | | | |
| (FP′) | % FPF | | MMAD (μ) | | GSD | | % FPF | | MMAD (μ) | | GSD | |
| 25/250 | SX | FP | SX | FP | SX | FP | SX | FP | SX | FP | SX | FP |
| Mean | 24.08 | 23.59 | 3.21 | 3.3 | 201 | 2.2 | 47.7 | 42.7 | 3.3 | 2.9 | 1.6 | 1.7 |
| SD | 1.67 | 1.14 | 0.09 | 0.1 | 0.03 | 0.1 | 2.0 | 4.1 | 0.06 | 0.1 | 0 | 0.0 |

TABLE 6b
Dry powder inhalation in market (prior art)
delivered via different inhalation devices

| Strength | | | | | | | | | | | | |
|---|---|---|---|---|---|---|---|---|---|---|---|---|
| Of | Prior art Composition | | | | | | Prior art Composition | | | | | |
| salmeterol | delivered via inhalation device | | | | | | delivered via | | | | | |
| (SX) and | available under the brand name | | | | | | applicant's patented | | | | | |
| fluticasone | of Rotahaler ® | | | | | | inhalation device | | | | | |
| (FP′) | % FPF | | MMAD (μ) | | GSD | | % FPF | | MMAD (μ) | | GSD | |
| 25/250 | SX | FP | SX | FP | SX | FP | SX | FP | SX | FP | SX | FP |
| Mean | 12.3 | 11.6 | 3.5 | 4.0 | 2.37 | 2.0 | 34.1 | 31.9 | 2.4 | 3.2 | 2.1 | 1.9 |
| SD | 1.51 | 1.8 | 0 | 0.1 | 0.1 | 0.0 | 1.8 | 1.4 | 0.1 | 0.04 | 0.01 | 0.02 |

The comparison of the % FPF, MMAD and GSD achieved by the dry powder composition of the present invention delivered by inhalation device available in market was found to superior compared to the % FPF, MMAD and GSD achieved by the dry powder composition available in the market delivered by inhalation device available in market. This is evident from the table 6a and table 6b. Particularly, the % FPF for salmeterol and fluticasone when present in the dry powder composition of the present invention was found to be 24.08, 23.59 compared to 12.27 and 11.60 when present in the composition of the prior art. The % FPF was further found to be improved when the composition of the present invention was delivered via the BAM inhalation device. It was found to be 47.68 and 42.74 for salmeterol xinafoate and fluticasone propionate, respectively. Thus the dry powder inhalation composition of the present invention that contains salmeterol xinafoate of specific tapped density, was found to show superior property when tested in-vitro using cascade impactor.

1. A dry powder inhalation composition comprising
(1) salmeterol xinafoate having mean particle size in range of 2.0μ-6μ microns and a tapped density in the range of 0.20 g·cm⁻³ to 0.45 g·cm⁻³ and
(2) optionally, one or more other active ingredients
and pharmaceutically acceptable carrier.

2. A dry powder inhalation composition as claimed in claim 1 wherein mean particle size of salmeterol xinafoate in about 4.0μ microns and a tapped density is 0.23 g·cm⁻³.

3. A dry powder inhalation composition as claimed in claim 1 wherein salmeterol xinafoate is in the form of polymorph I substantially free of polymorph II.

4. A dry powder inhalation composition as claimed in claim 3 wherein salmeterol xinafoate is obtained by a process comprising steps of
1. micronizing salmeterol xinafoate by milling
2. subjecting the micronized salmeterol xinafoate to a temperature of about 35° C. to 90° C. for a time period of about 1 hour to 120 hours, optionally, under pressure of about 1 to 100 bar.

5. A dry powder inhalation composition as claimed in claim 4 wherein the pressure is applied in presence of air or inert atmosphere of carbon dioxide or nitrogen.

6. A dry powder inhalation composition as claimed in claim 4 wherein the micronized salmeterol xinafoate is subjected to a temperature of about 40° C. and a pressure of about 80 bars, for a time period of about 80 hours, in carbon dioxide environment.

7. A method of treating asthma and other inflammatory respiratory disorders comprising administering by inhalation to humans in need of such treatment effective amounts of salmeterol xinafoate, wherein the method delivers higher amount of the inhaled drug to the lungs compared to the existing inhalation product.

8. A method of treating asthma and other inflammatory respiratory disorders comprising administering a dry powder inhalation composition comprising salmeterol xinafoate and fluticasone propionate and wherein the method provides equivalent efficacy of the inhaled active ingredient to the lungs at half the total dose in comparison to the existing inhalation product.

9. A method as claimed in claim 7, wherein the mean particle size of salmeterol xinafoate in range of 3.0μ-4μ microns and a tapped density in the range of 0.25 g·cm⁻³ to 0.35 g·cm⁻³.

10. A method as claimed in claim 9 wherein the salmeterol xinafoate is in the form of polymorph I substantially free of polymorph II.

11. A method as claimed in claim 8, wherein the mean particle size of salmeterol xinafoate in range of 3.0μ-4μ microns and a tapped density in the range of 0.25 g·cm⁻³ to 0.35 g·cm⁻³.

12. A method as claimed in claim 11 wherein the salmeterol xinafoate is in the form of polymorph I substantially free of polymorph II.
